(19) United States
(12) Patent Application Publication
(10) Pub. No.: US 2018/0317929 A1
(43) Pub. Date: Nov. 8, 2018

(54) Device and Method for Pre-Hospital Hemmorhage Control

(71) Applicants: Sean M. Brown, Pittsboro, NC (US); Andy Kiser, Pittsboro, NC (US)

(72) Inventors: Sean M. Brown, Pittsboro, NC (US); Andy Kiser, Pittsboro, NC (US)

(21) Appl. No.: 15/587,110

(22) Filed: May 4, 2017

(51) Int. Cl. A61B 17/12; A61B 17/122

(52) U.S. Cl. CPC A61B 17/12109; A61B 17/12031; A61B 2017/1205; A61B 17/122; A61B 17/12131

(57) ABSTRACT

A device and method to occlude blood flow through a subclavian artery and subclavian vein is presented. Small incisions are formed both superior and inferior to the clavicle. A closure element is introduced into one incision, passed posterior to the clavicle and vessels, and withdrawn through a second incision, such that a first end of the closure element extends from one incision and the second end extends from the other incision. The closure element is formed into a closed loop around the clavicle and vessels. The loop is tightened to occlude blood flow through the vessels. Locking features on the closure element enable rapid and secure cinching of the loop. A second closure element may be delivered through the same pathway and used for enhanced stability of the occlusion. A delivery member may be used to carry the closure element(s) through the incisions.

### PRIORITY CLAIM

This Non-Provisional application claims under 35 U.S.C. § 120, the benefit of priority the Provisional Application 62/331,983, filed May 5, 2016 titled “Device and Method for Pre-Hospital Hemorrhage Control” which is hereby incorporated by reference in its entirety.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

A large number of medical studies on the current Global War on Terrorism call for the development of new devices targeting the control of junctional hemorrhage. Junctional regions are those regions in which the extremities adjoin to the trunk, specifically the pelvic, neck, and axillary regions. These studies call for a device that specifically compresses the target tissues reliably and precisely¹ in junctional regions not amenable to traditional tourniquets, including specifically the axillary pocket.

The rapid development and increased use of tourniquets to treat distal extremity trauma in the pre-hospital setting increased survivability of these wounds by 90%.² However, for patients with proximal upper extremity or axillary pocket injuries, the use of such devices is precluded due to the anatomic location of the proximal blood supply (subclavian artery), which runs posterior to the clavicle in the upper chest. In today's battlefield, explosions account for 75% of all combat casualties.³ One study of blast fatalities estimated that approximately 22% were caused directly by junctional hemorrhage secondary to penetrating shrapnel.⁴ This illuminates a large percentage of combat casualties that would potentially benefit from an adaptation of the tourniquet to be used in junctional hemorrhage trauma. This application describes a closure element and delivery system that provides a safe and easy adaptation of the tourniquet that can treat axillary pocket and proximal upper extremity hemorrhage not amenable to the application of traditional tourniquet devices.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of the hemorrhage control device consistent with certain embodiments of the present invention.

FIG. 2 is a view of the hemorrhage control device locking portion consistent with certain embodiments of the present invention.

FIG. 3 is a view of the hemorrhage control device delivery jaw member structure consistent with certain embodiments of the present invention.

FIG. 4 is a view of the inner construction of the upper jaw member consistent with certain embodiments of the present invention.

FIG. 5 is a view of the open configuration for the hemorrhage control apparatus consistent with certain embodiments of the present invention.

FIG. 6 is a view of the closed and locked configuration for the hemorrhage control apparatus consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

For patients with non-compressible arterial and venous hemorrhage in the axillary pocket and proximal upper extremity, gaining control of the blood supply proximal to the site of trauma is necessary to prevent exsanguination, and ultimately death, in a field setting. In a hospital setting this could be done through invasive surgery to dissect and locate the source of bleeding and subsequently ligate the vessel. In a non-hospital setting, invasive surgery is contra-indicated due to lack of anesthesia and possible complications. Thus it is necessary to develop a new procedure that can be done in a non-hospital setting, requiring minimal sedation, and which poses a low risk of complication. Currently no device or procedure exists to specifically occlude the subclavian artery and vein under the aforementioned conditions. To perform the procedure and place a closure element, a small skin incision is made inferior to the clavicle. This incision is carried to the anterior ribcage via blunt dissection and then superiorly along this plane, posterior to the clavicle and vessels. Once the pulsation of the subclavian artery is palpated, the dissection is brought back up to the skin, superior to the clavicle, and a second small skin incision made over this projected exit site. The distal tip of the delivery element is then inserted into the inferior incision and passed through this tract, passing posterior to the clavicle and subclavian artery and vein, and exiting via the superior incision. Because the distal tip of the delivery element is blunt, it can pass between the pectoral muscle and the pectoral wall. Moreover, the smooth, continuous profile of the hinged portion of delivery element facilitates its passage through the tissue.

At this stage in the procedure, the free, “tail,” ends of the closure elements extend from the inferior incision and the delivery element extends from the superior incision. Once the attachment mechanism of the delivery element has exited the superior incision, the jaw members are opened and the distal end of the closure element is removed from the delivery element. The hinge is preferably oriented relative to the curvature of the delivery element such that the elements fall out from between the jaws as soon as the moveable jaw is moved to the open position.

The distal tip of the closure element may be passed through the locking eyelet on the tail of the closure element (or vice versa if the locking eyelet is on the distal tip) creating a closed loop around the bone, artery and vein. The closure element is then tightened to occlude the vessels to the point which hemorrhage is controlled. The user maintains a grip on the second closure element to maintain proximal control of the vessel. If the closure element breaks or fails to adequately control the hemorrhage, the second closure element may be locked in place (e.g. by tying knots), forming a secondary securing device to ensure stability during transport to higher care.

The delivery member and/or closure elements are preferably packaged with instructions for use describing the methods described herein. The package might additionally include a small scalpel for forming the incisions as well as a hemostat for opening the incision.

The embodiments shown in the drawings include attachment features that allow a user to attach the closure element(s) to the delivery member, and to easily separate the closure element(s) from the delivery member after they have been carried through the tract by the delivery member. This feature allows delivery member to be a reusable device that can be reloaded with fresh closure elements(s) after each use. A first responder might carry a delivery member, multiple sets of closures devices that can be loaded onto the delivery member, and means for disinfecting the delivery member between uses. It should be appreciated, however, that the system may be configured as a single use device, with the delivery member and closure element being integral components or permanently fixed to one another unless separated by cutting. In use of such an embodiment, the distal end of the delivery member may be advanced through a locking mechanism on the tail of the closure element once the delivery member has exited the superior incision, and then tightened as described above until the closure element is cinched against the bone, vein and artery. The locking mechanism may be a locking eyelet on the tail of the closure element, which engages with the locking ribs positioned on its shaft when it is cinched, or it may be some other form of locking mechanism that retains the cinched position of the closure element.

The disclosure herein provides a device, system and method of controlling bleeding by occluding blood vessels comprising a subclavian artery and subclavian vein, through the use of a closure element that may provide a cinch to close the subclavian artery and/or subclavian vein. The system and method is utilized by first forming a superior incision superior to a clavicle and forming an inferior incision inferior to the clavicle. The user may then introduce a first end of a closure element into a first one of the incisions, passing the closure element posterior to the clavicle and the vessels, and withdrawing the first end of the closure element through the second one of the incisions, such that the capture element extends between the superior and inferior incisions, with each end of the capture element extending from a different one of the incisions. The cinch may then be completed by forming a loop with the closure element to create a closed loop around the clavicle and vessels, and tightening the loop to occlude blood flow through the vessels.

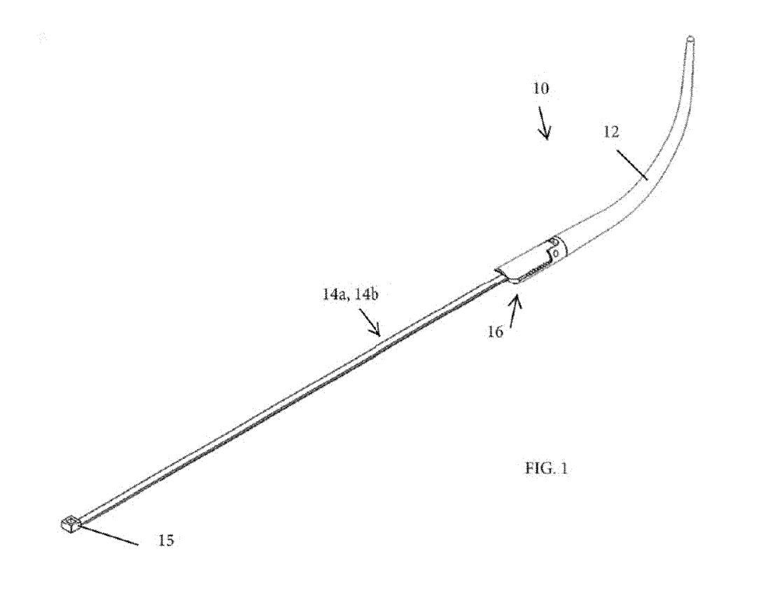

Turning now to FIG. 1, this figure presents a view of the hemorrhage control device consistent with certain embodiments of the present invention. In an exemplary embodiment, this figure presents a perspective view of the hemorrhage control device 10 utilizing principles of the present invention. The hemorrhage control device 10 includes a delivery member 12 and at least one closure element 14a carried by the device. Delivery member 12 is a rigid member having a distal end that is distally tapered to form a pointed tip. It is generally curved to allow insertion using a hooking motion, as will be appreciated from the description of use of use herein provided.

The closure element 14a is an elongate element formed of a material sufficiently flexible to allow it to be looped around, and tightened against, vascular tissue and bone as described below without severing the vascular tissue. The closure element 14a includes a locking mechanism (not shown) that allows the closure element 14a to be formed into a loop, cinched, and then locked in the looped and cinched configuration. The locking mechanism may function similar to the locking features on a “zip tie”. More specifically, a locking eyelet 15 similar to that found on a “zip tie” is positioned on a first end of the closure element 14a, and a collection of ribs (not shown) are formed near the second, opposite, end of the closure element 14a, so that when the second end is inserted into the locking eyelet 15 and pulled with sufficient force to cinch the closure element 14a, ribs pass through the locking eyelet during insertion but are unable to subsequently withdraw from the locking eyelet.

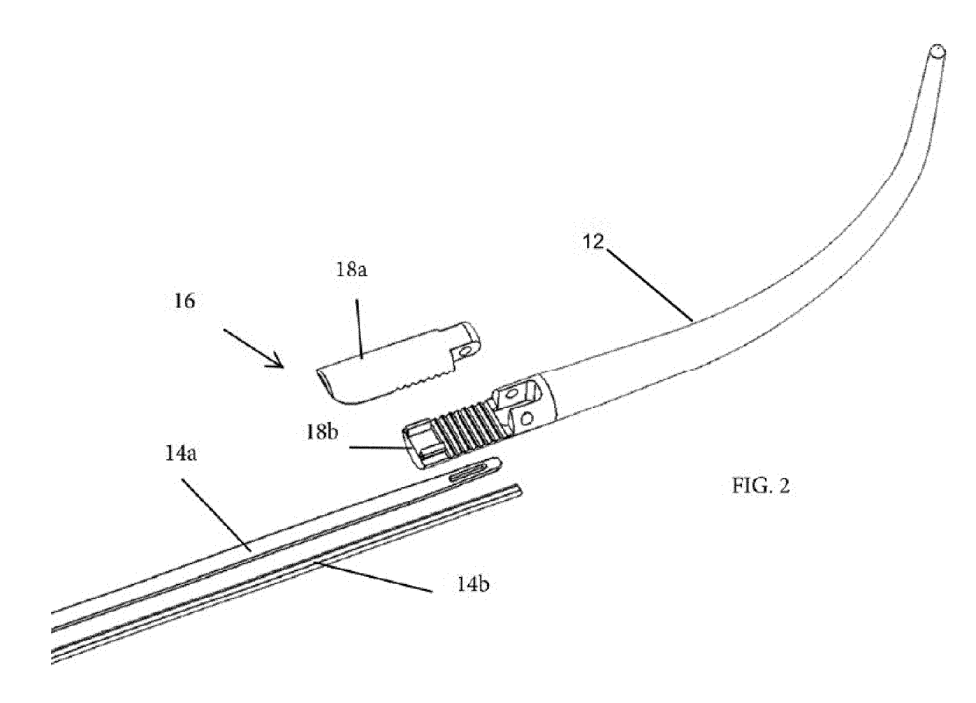

Turning now to FIG. 2, this figure presents a view of the hemorrhage control device locking portion consistent with certain embodiments of the present invention. In an embodiment, two closure elements 14a, 14b may be carried by the delivery member 12. The second closure element 14b may be similar to the first, or it may have a more simple construction. In one embodiment, the second closure element 14b may be a strand of suture material or umbilical tape, preferably having a flat or wide lateral cross-section to prevent it from having a sawing effect on the vascular tissue.

In this exemplary embodiment, the system presents an attachment mechanism 16 that may be located at the proximal end of the delivery member 12. The attachment mechanism 16 is used for removably attaching the closure element(s) to the delivery member 12. Various configurations can be used for the attachment mechanism 16. In an embodiment, the attachment mechanism 16 may be composed of an upper jaw member 18a and a lower jaw member 18b that fit together to enclose the distal end of the two closure elements 14a and 14b.

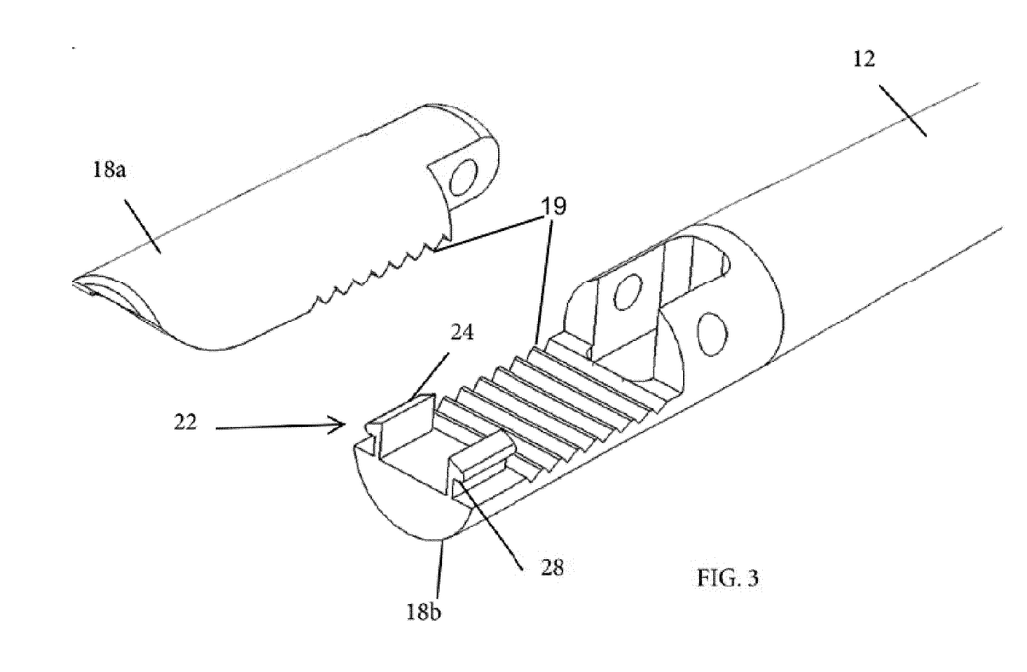

Turning now to FIG. 3, this figure presents a view of the hemorrhage control device delivery jaw member structure consistent with certain embodiments of the present invention. In an embodiment, a pair of jaw members, an upper jaw member 18a and a lower jaw member 18b, on the proximal part of the delivery member 12 may form a secure closure structure. The secure closure structure is operative to close around one or more closure elements (not shown) as the delivery member 12 is in operation. The lower jaw member 18b is a fixed jaw member, and the upper jaw member 18a is moveable at a hinge 20 relative to the fixed jaw member. In operation, the jaw members 18a, 18b are positioned in a closed position, with jaw member 18a in full contact with jaw member 18b, to grasp the distal ends of the closure elements (not shown) between the two jaw members 18a, 18b. The jaw members 18a, 18b, have opposed grasping surfaces with surface features 19 that facilitate secure retention of the closure elements (not shown) between the jaws 18a, 18b. In a non-limiting example, the surface features 19 may be serrations that enmesh with one another when the jaws 18a, 18b are closed, securely clamping the closure elements between them. However, it may be understood that additional surface features 19 may be implemented, such as additional interlocking geometric shapes, flat surfaces with grooved sections for the closure elements, dovetail surfaces that fit together, or any other configuration that forms a secure clamping element between the upper jaw 18a and the lower jaw 18b.

In an exemplary embodiment, the delivery member may include a latch configured to securely engage the jaw members 18a, 18b in the closed position, ensuring that the closure elements will remain held by the jaws until the user takes steps to remove them. In the drawings, the latch includes a pair of spring members 22 on one of the jaws 18b and recesses on the upper jaw 18a. The spring members 22 have beveled surfaces 24 and catch elements 28. As the jaws are closed, contact between the beveled surfaces 24 and the walls of the recesses presses the spring members 22 laterally inwardly, against their natural bias. Once the catch elements 28 pass into the recesses, the spring members 22 spring laterally outward in accordance with their bias, causing their catch elements 28 to be captured by interior shoulders within the recesses, and latching the jaw members in the closed position.

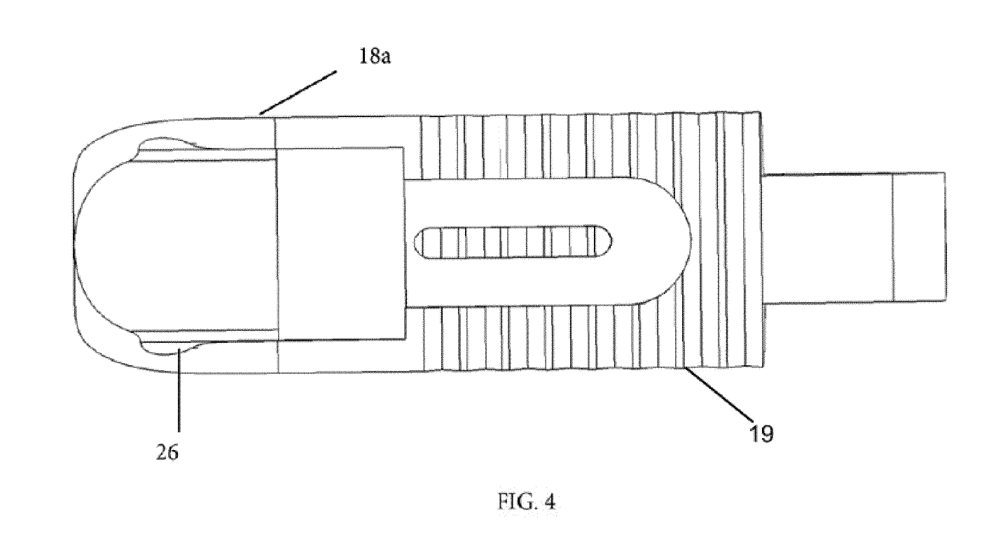

Turning now to FIG. 4, this figure presents a view of the inner construction of the upper jaw member consistent with certain embodiments of the present invention. In an exemplary embodiment, the upper jaw 18a may have surface features 19 that provide for secure capture of the closure elements (not shown) when the upper jaw 18a is in contact with the lower jaw 18b (as shown in FIG. 3). The upper jaw 18a may also have recesses 26 positioned to make contact with the catch elements (not shown). Upon contact with the catch elements, the recesses 26 are configured to permit the catch elements to push into the recesses 26 when the jaw elements are in sufficient contact, causing the catch elements to be captured by the recesses 26 and latching the jaw members in the closed position.

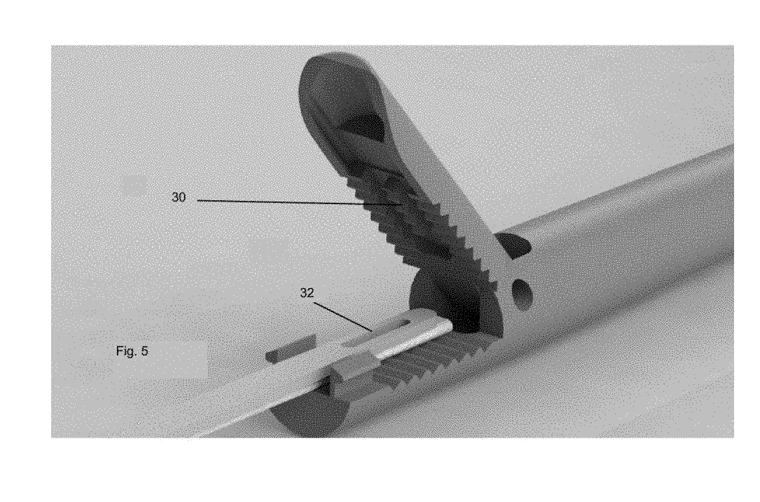

Turning now to FIG. 5, this figure presents a view of the open configuration for the hemorrhage control apparatus consistent with certain embodiments of the present invention. In this embodiment, the upper jaw element 18a is in an unlocked and open position, swiveling vertically away from the lower jaw element 18b to permit one or more closure elements to be inserted within the jaw construction. As previously described, the upper jaw element 18a and the lower jaw element 18b each comprise a surface feature constructed to fit together when the jaw element are closed to secure the closure. In this exemplary embodiment, a tab feature 30 is formed in the center of the upper jaw element 18a. The tab feature 30 is positioned such that the tab feature 30 will be inserted into a window portion 32 of the closure element to secure the closure element within the jaws. In additional exemplary embodiments, other types of surface patterns or adhesive surfaces may be used to secure the closure element within the jaws when the jaw elements are in the closed position. Additionally, the closure elements might be provided with adhesive tabs at their distal ends that may be adhered to either or both of the grasping surfaces of the jaws.

In an alternative embodiment, rather than using serrations to retain the one or more closure elements, the jaw structure may consist of a post on one of the jaws elements. In this exemplary embodiment, each closure element may include an eyelet on the end of the closure element positioned within the jaw structure. The eyelets on the one or more closure elements may be positioned over the post when the jaws are in the opened position. The jaws may then be moved to a closed position to prevent the one or more closure elements from slipping off the post.

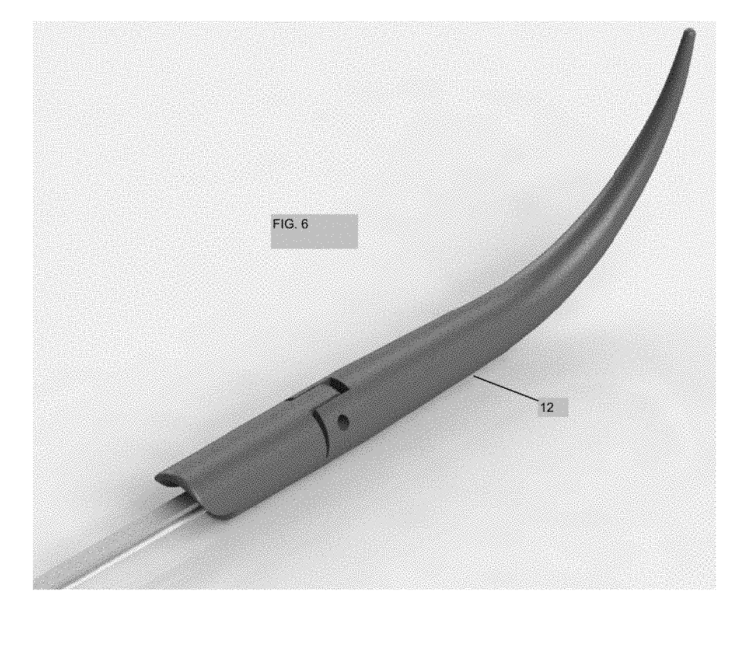

Turning now to FIG. 6, this figure presents a view of the closed and locked configuration for the hemorrhage control apparatus consistent with certain embodiments of the present invention. In this embodiment, the jaw elements 18a, 18b are closed and locked, securing the closure elements within the jaw elements. Additionally, the delivery element 12 may have gripping features on its surface that allow the user to maintain a tight grasp on the delivery element 12 while withdrawing it from the tissue, but that do not significantly impede its passage through the tract. Such features may be conical barbs that are oriented with their narrow ends towards the distal tip. When closed and locked, securing one or more closure elements within the jaw elements, the hemorrhage control apparatus is prepared for use.

While certain illustrative embodiments have been described, it is evident that many alternatives, modifications, permutations and variations will become apparent to those skilled in the art in light of the foregoing description.

We claim:

1. A device for controlling bleeding by occluding blood vessels comprising a subclavian artery and subclavian vein, comprising:
a delivery member having a tapered shape and an openable jaw portion;
said delivery member jaw portion associated with one or more closure elements, to which said one or more closure elements are releasably attached;
forming a superior incision superior to a clavicle and forming an inferior incision inferior to the clavicle;
introducing a first end of a closure element into a first one of the incisions by passing said delivery member through said incisions, passing the closure element posterior to the clavicle and the vessels, and withdrawing the first end of the closure element through the second one of the incisions, such that the capture element extends between the superior and inferior incisions, with each end of the capture element extending from a different one of the incisions;
forming a loop with the closure element to create a closed loop around the clavicle and vessels, and tightening the loop to occlude blood flow through the vessels.

2. The device of claim 1, wherein:
the first end of the closure element is attached to a delivery member; and
introducing the delivery member into the first one of the incisions and passing the delivery member posterior to the clavicle and vessels to draw the closure element posterior to the clavicle and vessels.

3. The device of claim 2, where, prior to forming the loop, the closure element is detached from the delivery member and a distal end of said closure element being passed through a locking end of said closure element to form a loop that is locked in place by said locking end.

4. The device of claim 1, further including locking the closure element in the tightened position to sustain the occlusion of blood flow.

5. The device of claim 2, wherein:
a second closure element is attached to the delivery member;
introducing the delivery member into the first one of the incisions and passing the delivery member posterior to the clavicle and vessels draws the second closure element posterior to the clavicle and vessels; and using the second closure element to maintain proximal control of the vessel.

6. The device of claim 5, further including forming a second loop with the second closure element to create a closed second loop around the clavicle and vessels, tightening the second loop against the vessels, and locking said second loop in place.

7. A method for controlling bleeding by occluding blood vessels comprising a subclavian artery and subclavian vein, comprising:
selecting a closure element having a first end and a second end;
instructions for use instructing a user to:
form a superior incision superior to a clavicle and an inferior incision inferior to the clavicle;
introduce a first end of a closure element into a first one of the incisions, pass the closure element posterior to the clavicle and the vessels, and withdraw the first end of the closure element through the second one of the incisions, such that the capture element extends between the superior and inferior incisions, with each end of the capture element extending from a different one of the incisions;
form a loop with the closure element to create a closed loop around the clavicle and vessels, and
tighten the loop to occlude blood flow through the vessels.

8. A system for controlling bleeding by occluding blood vessels comprising a subclavian artery and subclavian vein, comprising:
a closure element having a locking mechanism;
a delivery member having a distal tip and a proximal portion, the closure element removably attachable to the proximal portion; and
forming a superior incision superior to a clavicle and an inferior incision inferior to the clavicle;
with the closure element attached to the delivery member, introduce the distal tip of the delivery member into a first one of the incisions, pass the delivery member and closure element posterior to the clavicle and the vessels, and withdraw the distal tip of the closure element through the second one of the incisions, carrying a portion of the capture element out of the second one of the incisions, while a tail of the capture element extends from the first one of the incisions;
forming a loop with the closure element to create a closed loop around the clavicle and vessels, and
tightening the loop to occlude blood flow through the vessel, and engage the loop in the tightened position using the locking mechanism.

9. The system of claim 8, wherein the delivery member has proximal jaws, and wherein the capture element is attachable to the delivery member by positioning the jaws in an opened position, placing an end of the capture element between the jaws, and moving the jaws to the closed position.

10. The system of claim 9 wherein the jaws include a latch for retaining the jaws in the closed position.

11. The system of claim 9, wherein the jaws include serrations for gripping the capture element when the jaws are in the closed position.

12. The system of claim 9 wherein one of the jaws includes a post, and the capture element includes an eyelet mountable on the post.
